(19) United States
(12) Patent Application Publication
(10) Pub. No.: US 2013/0226066 A1
(43) Pub. Date: Aug. 29, 2013

(54) Apparatus for monitoring cerebrospinal fluid drainage

(71) Applicant: Jung-Tung Liu, Taichung (TW)

(72) Inventor: Jung-Tung Liu, Taichung (TW)

(21) Appl. No.: 13/402,905

(22) Filed: Feb. 23, 2012

(51) Int. Cl. A61M 1/00

(52) U.S. Cl. 604/9

(57) ABSTRACT

An apparatus for monitoring CSF drainage is provided with a tubing having one end installed in a brain of a patient and the other end installed in an abdominal cavity of the patient for draining CSF from the brain to the abdominal cavity; an internal monitoring device disposed in the tubing proximate to the abdominal cavity, the internal monitoring device being for measuring fluid pressure of the CSF and regulating flow rate of the CSF; and an external processing device disposed externally of the patient, the external processing device being for wirelessly controlling the internal monitoring device by processing a fluid pressure signal and a flow rate signal both transmitted from the internal monitoring device, and by transmitting both a fluid pressure control signal and a flow rate control signal to the internal monitoring device. The external processing device includes a display for displaying processed data.

### BACKGROUND OF THE INVENTION

1. Field of the Invention

The invention relates to shunt systems for draining cerebrospinal fluid within a patient and more particularly to an apparatus for monitoring cerebrospinal fluid drainage.

2. Description of Related Art

Cerebrospinal fluid (CSF) is a clear, colorless, bodily fluid that occupies the subarachnoid space and the ventricular system around and inside the brain and spinal cord. The CSF occupies the space between the arachnoid mater and the pia mater. It constitutes the content of all intra-cerebral ventricles, cisterns, and sulci as well as the central canal of the spinal cord.

CSF is produced in the brain and reabsorbed into venous sinus blood via arachnoid granulations. CSF is produced at a rate of 500 ml/day. Since the brain can contain only 135 to 150 ml, large amounts are drained primarily into the blood through arachnoid granulations in the superior sagittal sinus. Thus the CSF turns over about 3.7 times a day. This continuous flow into the venous system dilutes the concentration of larger, lipoinsoluble molecules penetrating the brain and CSF.

CSF serves four primary purposes: Buoyancy, protection, chemical stability, and prevention of brain ischemia. When CSF pressure is elevated, cerebral blood flow may be constricted. When disorders of CSF flow occur, they may therefore affect not only CSF movement but also craniospinal compliance and the intracranial blood flow, with subsequent neuronal and glial vulnerabilities.

Hydrocephalus is a condition afflicting patients who are unable to regulate cerebrospinal fluid flow through their body's own natural pathways. Produced by the ventricular system, CSF is normally absorbed by the body's venous system. In a patient suffering from hydrocephalus, the CSF is not absorbed in this manner, but instead accumulates in the ventricles of the patient's brain. If left untreated, the increasing volume of fluid elevates the patient's intracranial pressure and can lead to serious medical conditions such as subdural hematoma, compression of the brain tissue, and impaired blood flow.

Treatment of hydrocephalus has conventionally involved draining the excess fluid away from the ventricles and rerouting the cerebrospinal fluid to another area of the patient's body, such as the abdomen or vascular system. A drainage system, typically referred to as a shunt, is often used to carry out the transfer of fluid. A typical Ventriculo-Peritoneal Shunt system for draining fluid within a patient comprises a proximal catheter installed in the ventricular cavity of the patient's brain, a distal catheter installed in a portion (e.g., ventricle) of the patient's body where the excess fluid is to be reintroduced, a tubing interconnecting the catheters, and a flow control valve adjacent to the proximal catheter. The flow control valve can close or open based on predetermined pressure settings so as to adjust the volume of CSF flowing to the abdominal cavity.

However, the typical Ventriculo-Peritoneal Shunt system is not capable of measuring real time flow and volume of the CSF flow. Doctors do not rely much on the typical shunt systems when conducting a surgery or examining a patient. Thus, the need for improvement still exists.

### SUMMARY OF THE INVENTION

It is therefore one object of the invention to provide an apparatus for monitoring CSF drainage comprising a tubing having one end installed in a brain of a patient and the other end installed in an abdominal cavity of the patient for draining CSF from the brain to the abdominal cavity; an internal monitoring device disposed in the tubing proximate to the abdominal cavity, the internal monitoring device being for measuring fluid pressure of the CSF and regulating flow rate of the CSF; and an external processing device disposed externally of the patient, the external processing device being for wirelessly controlling the internal monitoring device by processing a fluid pressure signal and a flow rate signal both transmitted from the internal monitoring device, and by transmitting both a fluid pressure control signal and a flow rate control signal to the internal monitoring device, the external processing device comprising a display for displaying processed data.

The above and other objects, features and advantages of the invention will become apparent from the following detailed description taken with the accompanying drawings.

### BRIEF DESCRIPTION OF THE DRAWINGS

FIG. 1 is an environmental view of an apparatus for monitoring cerebrospinal fluid drainage according to the invention;

FIG. 2 is a block diagram of the internal monitoring device and the external processing device; and

FIG. 3 is an enlarged view of the upper portion of FIG. 1.

### DETAILED DESCRIPTION OF THE INVENTION

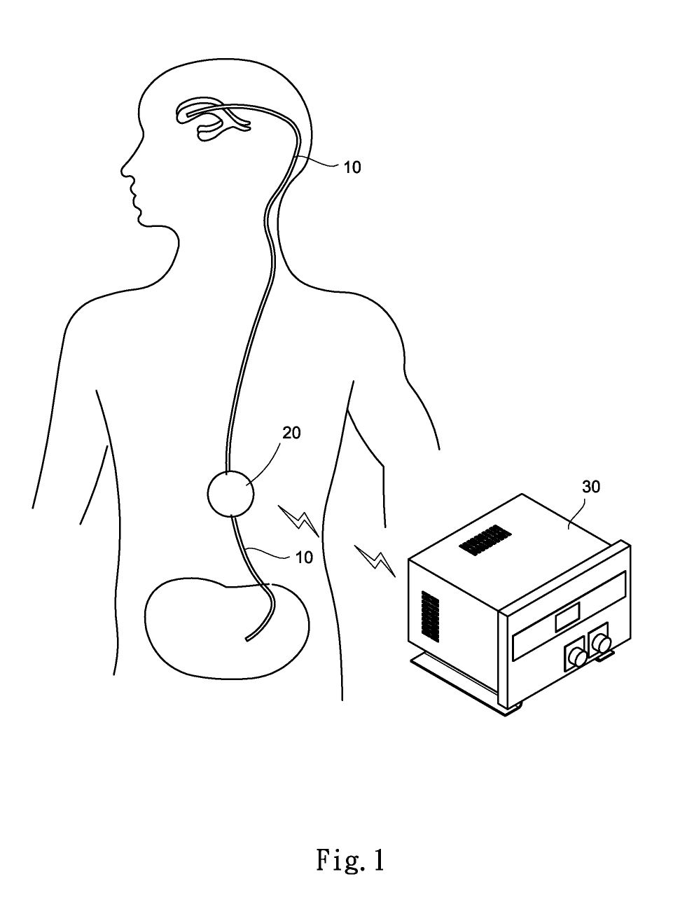

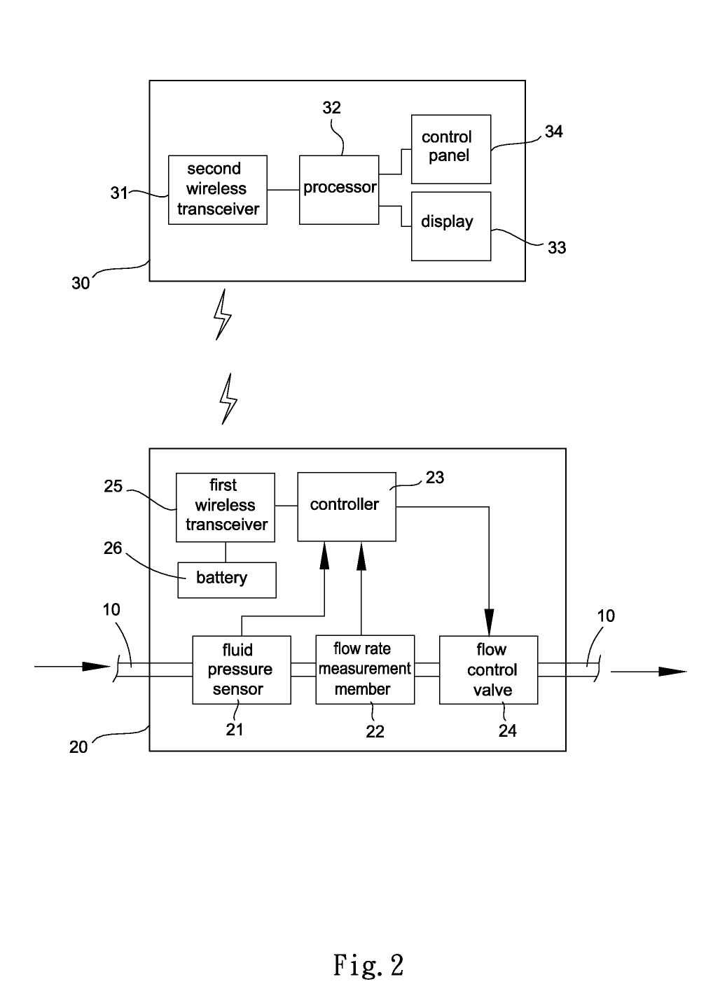

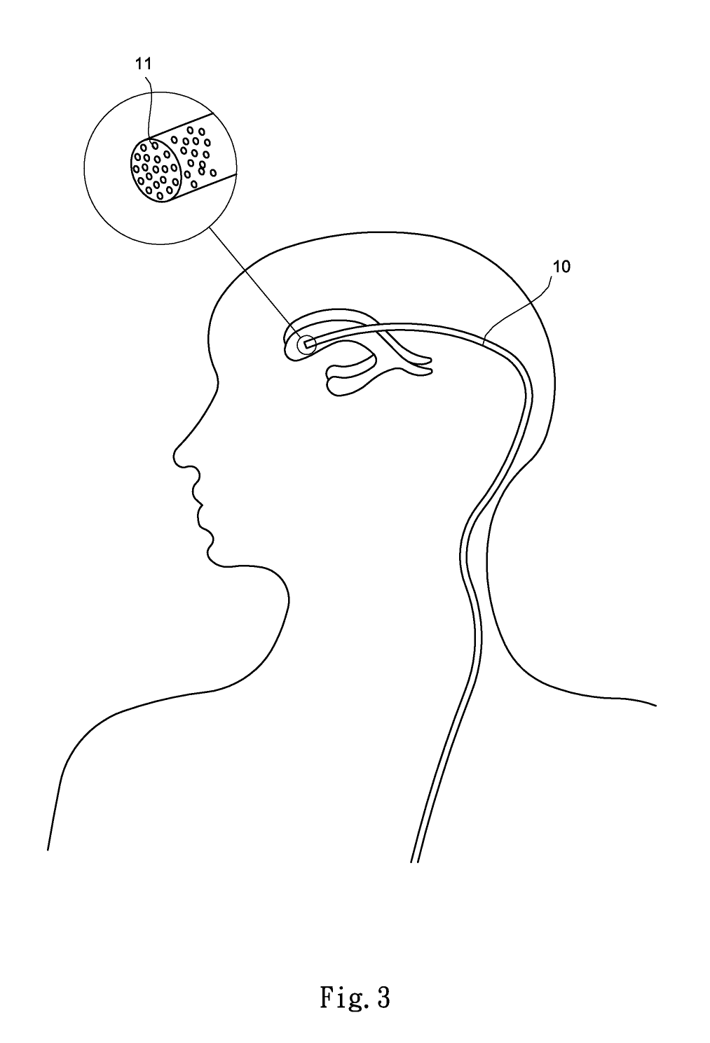

Referring to FIGS. 1 to 3, an apparatus for monitoring cerebrospinal fluid (CSF) drainage in accordance with the invention comprises the following components as discussed in detail below.

A tubing 10 has one end formed as a first catheter installed in the brain and the other end formed as a second catheter installed in an abdominal cavity. The first catheter is comprised of a plurality of holes 11 for blocking large particles of the CSF from entering the tubing 10. The second catheter can reintroduce the CSF to the abdominal cavity.

An internal monitoring device 20 is disposed in a predetermined position of the tubing 10 proximate to the abdominal cavity and comprises a fluid pressure sensor 21 for sensing fluid pressure of the CSF flowing through the tubing 10, a flow rate measurement member 22 for measuring the flow rate of the CSF flowing through the tubing 10, and a flow control valve 24 for regulating the CSF flow. Above three components are disposed along the tubing 10.

The internal monitoring device 20 further comprises a controller 23 for receiving an analog fluid pressure signal from the fluid pressure sensor 21 and converting same into a digital fluid pressure signal, receiving an analog flow rate signal of the CSF and converting same into a digital flow rate of the CSF, and issuing instructions to the flow control valve 24 for closing the flow control valve 24 or opening same with different opening degrees based on the flow rate of the CSF, a first wireless transceiver 25 which is an RF (radio frequency) based member and is capable of receiving signals including fluid pressure and flow rate signals from the controller 23, transferring the signals by using radio waves, and receiving wireless signals from a signal source externally of a patient, and a battery 26 for supplying power to both the controller 23 and the first wireless transceiver 25.

An external processing device 30 is disposed externally of a patient and comprises a second wireless transceiver 31 which is also an RF based member and is capable of receiving signals including fluid pressure and flow rate signals from the first wireless transceiver 25, a processor 32 for receiving the digital signals from the second wireless transceiver 31, processing same into a fluid pressure value and a flow rate value, the fluid pressure value and the flow rate value being compared with a predetermined (i.e., safe) fluid pressure and a predetermined flow rate respectively, and wirelessly transferring controlled fluid pressure and flow rate signals to the first wireless transceiver 25, a display (e.g., liquid crystal display (LCD) display) 33 for displaying the measured fluid pressure, flow rate and other operation information in real time, and a control panel 34 including a plurality of buttons for medical employees operation so that a medical employee may remotely control the internal monitoring device 20 by pressing the buttons on the control panel 34 and watching data shown on the display 33. That is, a medical employee can control the open degree of the flow control valve 24 via a signal transmission from the processor 32, the second wireless transceiver 31, the first wireless transceiver 25, and the controller 23. As a result, a precise regulation of the CSF flow from the brain to the abdominal cavity can be achieved.

While the invention has been described in terms of preferred embodiments, those skilled in the art will recognize that the invention can be practiced with modifications within the spirit and scope of the appended claims.

1. An apparatus for monitoring CSF drainage comprising:
a tubing having one end configured for installation in a brain of a patient and the other end installed in an abdominal cavity of the patient for draining CSF from the brain to the abdominal cavity;
an internal monitoring device disposed in the tubing proximate to the abdominal cavity, the internal monitoring device being for measuring fluid pressure of the CSF and regulating flow rate of the CSF; and
an external processing device disposed externally of the patient, the external processing device being for wirelessly controlling the internal monitoring device by processing a fluid pressure signal and a flow rate signal both transmitted from the internal monitoring device, and by transmitting both a fluid pressure control signal and a flow rate control signal to the internal monitoring device, the external processing device comprising a display for displaying processed data;
wherein one end of the tubing is formed with a plurality of holes;
wherein the internal monitoring device comprises a fluid pressure sensor for sensing fluid pressure of the CSF, a flow rate measurement member for measuring flow rate of the CSF, a flow control valve for regulating flow of the CSF flow, a controller for receiving an analog fluid pressure signal from the fluid pressure sensor and converting same into a digital fluid pressure signal, receiving an analog flow rate signal of the CSF and converting same into a digital flow rate signal of the CSF, and issuing instructions to the flow control valve for closing the flow control valve or opening same with different opening degrees based on the flow rate of the CSF, an RF first wireless transceiver for receiving the digital fluid pressure signal and the digital flow rate signal from the controller, and transferring the digital fluid pressure signal and the digital flow rate signal, and a battery for supplying power to both the controller and the RF first wireless transceiver;
wherein the external processing device further comprises an RF second wireless transceiver for receiving the digital fluid pressure signal and the digital flow rate signal from the RF first wireless transceiver, a processor for receiving the digital fluid pressure signal and the digital flow rate signal from the RF second wireless transceiver, processing same into a fluid pressure value and a flow rate value, and wirelessly transferring a processed fluid pressure signal and a processed flow rate signal to the RF first wireless transceiver, and a control panel for manual operation to remotely control an open degree of the flow control valve; and
wherein the fluid pressure value and the flow rate value are compared with a predetermined fluid pressure and a predetermined flow rate respectively by the processor, and wherein the control panel is operative to remotely control the open degree of the flow control valve via the processor, the RF second wireless transceiver, the RF first wireless transceiver, and the controller.

2-5. (canceled)
